(19) United States
(12) Patent Application Publication
(10) Pub. No.: US 2022/0403450 A1
(43) Pub. Date: Dec. 22, 2022

(54) SYSTEMS AND METHODS FOR SEQUENCING NUCLEOTIDES USING TWO OPTICAL CHANNELS

(71) Applicants: Illumina Software, Inc., San Diego, CA (US); Illumina Cambridge Limited, Cambridge (GB)

(72) Inventors: Xiaohai Liu, Cambridge (GB); Michael Callingham, Saffron Walden (GB); Robert Ezra Langlois, San Diego, CA (US); Patrick McCauley, Linton (GB)

(21) Appl. No.: 17/338,590

(22) Filed: Jun. 3, 2021

(51) Int. Cl. C12Q 1/6825; G01N 21/64

(52) U.S. Cl. CPC C12Q 1/6825; G01N 21/6428; G01N 2021/6441

(57) ABSTRACT

The disclosed technology relates to the field of nucleic acid sequencing, and more particularly, to systems and methods for DNA sequencing utilizing a single optical excitation and at least three fluorescent labels. In some embodiments, the disclosed technology uses a first nucleotide coupled to a first fluorescent label which can emit light to be detectable by a first detector, a second nucleotide coupled to a second fluorescent label which can emit light to be detectable by a second detector, a third nucleotide coupled to a third fluorescent label which can emit light to be detectable by both the first and second detectors, and a fourth nucleotide coupled to no fluorescent label. The disclosed technology may identify a nucleotide in the nucleic acid sequence based on whether the emission is received by the first detector, the second detector, both the first and second detectors, or neither the first nor second detector.

### BACKGROUND

In some types of next-generation sequencing technologies, DNA clusters are created on a flowcell following amplification of a target polynucleotide. Increasing DNA cluster density within the flowcells (e.g. via the use of nanowells) and deploying faster imaging technologies can scale up DNA sequencing throughput and reduce overall sequencing costs. However, the use of faster imaging technologies can lead to the signal from DNA clusters becoming dimmer, and higher power light sources being required to compensate for the dimmer signal. High power light sources, such as high power lasers may be expensive, consume relatively high amounts of energy, and generate a substantial amount of heat that needs to be dissipated. Furthermore, higher power light exposure may cause more light-induced damage to the target polynucleotide leading to a faster signal decay and reduced sequencing data quality over many sequencing cycles.

### SUMMARY

Existing DNA sequencing systems and methods, e.g., existing sequencing platforms using two or four-channel sequencing chemistry, may utilize two or more excitation light sources to excite deoxyribonucleic acid analogs conjugated with fluorescent labels in a target polynucleotide. Reducing the number of excitation light sources may reduce the cost and increase the performance robustness of such sequencing systems. In addition, reducing the number of excitation light sources may reduce unnecessary exposure of the samples to light, thus reducing light-induced DNA damage.

In one aspect, disclosed is a system for identifying a nucleotide in a polynucleotide bound to a substrate and a method of using such system. The system may include a first detector configured to detect a first range of wavelengths of light; a second detector configured to detect a second range of wavelengths of light; a light source comprising a laser or a light-emitting diode which outputs light at an optical frequency; and a processor. The processor may be configured to: generate light at the optical frequency to stimulate an emission from the nucleic acid sequence on the substrate; and identify a nucleotide in the nucleic acid sequence based on whether the emission is received by the first detector, the second detector, both the first and second detectors, or neither the first nor second detector. The system may further include a first nucleotide coupled to a first fluorescent label; a second nucleotide coupled to a second fluorescent label; a third nucleotide coupled to a third fluorescent label; and a fourth nucleotide coupled to no fluorescent label. The light source may be configured to: excite the first fluorescent label to emit light to be detectable by the first detector; excite the second fluorescent label to emit light to be detectable by the second detector; and excite the third fluorescent label to emit light to be detectable by both the first and second detectors.

Another embodiment is a method for determining the sequence of a polynucleotide that includes: emitting light at an optical frequency from a light source onto a polynucleotide; determining if the polynucleotide has a bound fluorescent label which fluoresces at a first wavelength of light, a second wavelength of light, both the first and second wavelengths of light, or has no fluorescence; and identifying the sequence of the polynucleotide based on whether there is a detectable emission at the first wavelength of light, the second wavelength of light, both the first and second wavelengths of light, or has no fluorescence.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

It is to be understood that any features of the systems disclosed herein may be combined together in any desirable manner and/or configuration. Further, it is to be understood that any features of the methods disclosed herein may be combined together in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates an example sequencing system which can perform embodiments of the disclosed sequencing technology.

FIG. 1B schematically illustrates an example imaging system to be used in embodiments of the disclosed sequencing technology.

FIG. 1C schematically illustrates another example imaging system to be used with embodiments of the disclosed sequencing technology.

FIG. 2 shows a functional block diagram of an example computer system to be used in the sequencing system as shown in FIG. 1A.

FIG. 3 shows an example dye labeling scheme for embodiments of the disclosed sequencing technology.

FIG. 4 shows example emission spectra of a collection of fully-functionalized nucleotides within embodiments of the disclosed sequencing technology.

FIG. 5 schematically illustrates an example of the fluorescent results from a single excitation, two-optical channel detection of three fully-functionalized nucleotides.

FIG. 6 shows the scatterplot results of a sequencing experiment performed according to one embodiment of the disclosed technology

FIG. 7A and FIG. 7B are line graphs showing the results of a sequencing experiment performed according to one embodiment of the disclosed technology.

FIG. 8 shows the scatterplot results of an additional sequencing experiment performed according to one embodiment of the disclosed technology.

FIG. 9A shows the scatterplot results of an alternative additional sequencing experiment performed according to an embodiment of the disclosed technology

FIG. 9B and FIG. 9C are line graphs showing the results of an alternative additional sequencing experiment performed according to and embodiment of the disclosed technology.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### Introduction

Embodiments of the disclosed technology relate to next-generation sequencing systems and methods that can identify four nucleotide bases using a single excitation light source and two different optical channels. The disclosed sequencing technology can make use of a sequencing-by-synthesis process. During each sequencing cycle, four types of nucleotide analogs can be incorporated onto growing primers hybridized to polynucleotides being sequenced. In some embodiments, the four types of nucleotide analogs can include a deoxyguanosine triphosphate (dGTP) analog not conjugated with any fluorescent dye, a deoxythymidine triphosphate (dTTP) analog conjugated with a first fluorescent dye, a deoxycytidine triphosphate (dCTP) analog conjugated with a second fluorescent dye, and a deoxyadenosine triphosphate (dATP) analog conjugated with a third fluorescent dye. The fluorescent dyes conjugated to the four types of nucleotide analogs are illustrative only, and not intended to be limiting. In other embodiments, the nucleotide analog not conjugated with any fluorescent dye may be dTTP, dCTP, or dATP. In other embodiments, the nucleotide analog conjugated with the first fluorescent dye may be dGTP, dCTP, or dATP. In other embodiments, the nucleotide analog conjugated with the second fluorescent dye may be dGTP, dTTP, or dATP. In other embodiments, the nucleotide analog conjugated the third fluorescent dye may be dGTP, dTTP, or dCTP.

The three fluorescent dyes can be excited by a single wavelength (or a single narrow band of wavelengths) of excitation light from a light source, such as a laser. The first fluorescent dye has an emission spectrum that can be captured in a first image taken in a first optical channel. The second fluorescent dye has an emission spectrum that can be captured in a second image taken in a second optical channel. The third fluorescent dye has an emission spectrum which is broad enough to be captured in images captured from both the first and second optical channels. Therefore, a nucleotide analog (or a DNA cluster having a plurality of the same nucleotide analog) associated with no dye, the first dye, the second dye, or the third dye can be identified based on whether a diffraction-limited spot occurs in no image, the first image, the second image, or both images, respectively.

Non-limiting advantages of the disclosed systems and methods include allowing a more efficient sequencing workflow with fewer process steps. In addition, sequencing systems which use a three-dye system as described herein may have fewer components and be less costly to operate and more power-efficient. For example, the disclosed systems and methods may require fewer numbers of excitation light sources than prior systems. In some embodiments, only a single excitation light source may be required, compared to prior system which required multiple excitation light sources. This leads to fewer necessary imaging steps and may enable the system to be more power-efficient. Having fewer components in a sequencer also may result in a substantial cost reduction and a simpler instrument design. Having fewer components in a sequencer may also increase the efficiency of the system and the robustness of instrument performance. In addition, the disclosed systems and methods may require a lower exposure of the target polynucleotide to the excitation light, which can alleviate light-induced DNA damage and therefore increase sequencing data quality and sequence base-calling accuracy.

### Example Sequencer

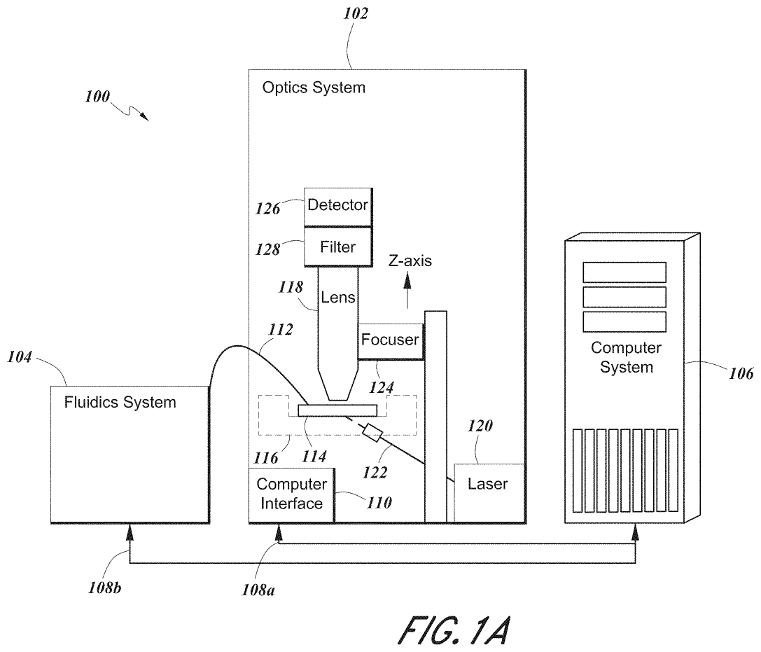

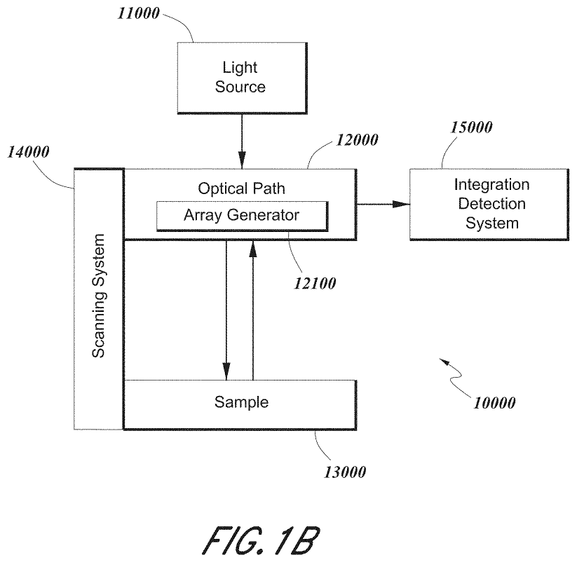

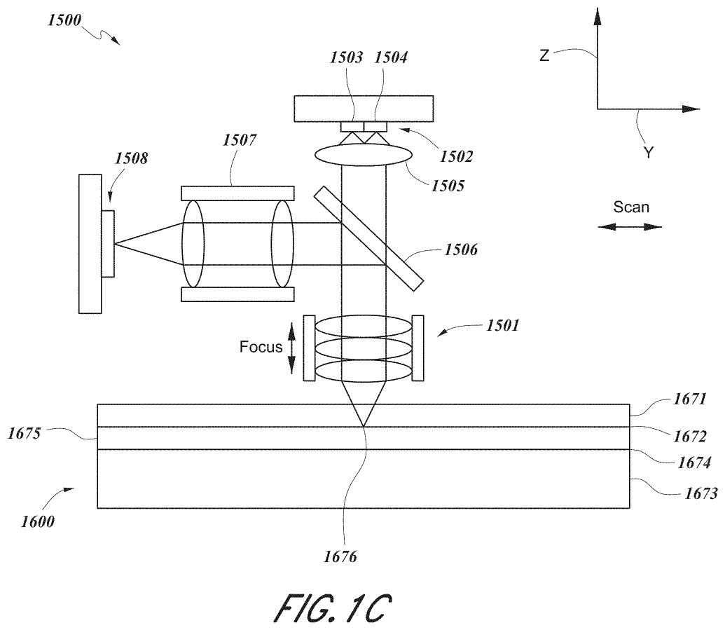

In FIG. 1A, an example sequencing system 100 which can perform the disclosed sequencing technology is illustrated. The sequencing system 100 can be configured to utilize disclosed sequencing methods based on a single optical excitation and at least three fluorescent labels. Non-limiting examples of the sequencing reactions utilized can include variations of sequencing-by-synthesis processes, such as those used in Illumina® dye sequencing or HeliScope® single molecule sequencing.

The sequencing system 100 can include an optics system 102 configured to generate raw sequencing data using sequencing reagents supplied by a fluidics system 104 that is part of the sequencing system 100. The raw sequencing data can include fluorescent images captured by the optics system 102. The sequencing system 100 can further include a computer system 106 that can be configured to control the optics system 102 and the fluidics system 104 via communication channels 108a and 108b. For example, a computer interface 110 of the optics system 102 can be configured to communicate with the computer system 106 through the communication channel 108a.

During sequencing reactions, the fluidics system 104 can direct the flow of reagents through one or more reagent tubes 112 to and from a flowcell 114 positioned on a mounting stage 116. The reagents can include, for example, fluorescently labeled nucleotides, buffers, enzymes, and cleavage reagents. The flowcell 114 can include at least one fluidic channel. The flowcell 114 can be a patterned array flowcell or a random array flowcell. The flowcell 114 can include multiple clusters of single-stranded polynucleotides to be sequenced in the at least one fluidic channel. The lengths of the polynucleotides can vary ranging, for example, from 200 bases to 1000 nucleotides. The polynucleotides can be attached to one or more fluidic channels of the flowcell 114. In some embodiments, the flowcell 114 can include a plurality of wells, wherein each well can include multiple copies of a polynucleotide to be sequenced. The mounting stage 116 can be configured to allow proper alignment and movement of the flowcell 114 in relation to the other components of the optics system 102. In one embodiment, the mounting stage 116 can be used to align the flowcell 114 with a lens 118.

The optics system 102 can include a single light source 120, such as a single laser or a single LED, configured to generate light having wavelengths narrowly distributed at around a predetermined wavelength, for example 455 nm. In some embodiments, the predetermined wavelength is within the range of 405 nm-460 nm. However, embodiments are not limited to any particular wavelength of light. The light source only needs to be configured to generate the correct wavelength of light which excites the fluorescent labels attached to the nucleotides on the flowcell.

The light generated by the light source 120 can pass through a fiber optic cable 122 to excite fluorescent labels in the flowcell 114. The lens 118, mounted on a focuser 124, can move along the z-axis. The focused fluorescent emissions can be detected by a detector 126, for example a charge-coupled device (CCD) sensor or a complementary metal oxide semiconductor (CMOS) sensor. In some embodiments, nucleotide incorporations can be detected with zeromode waveguides as described, for example, in Levene et al. Science 299, 682-686 (2003); Lundquist et al. Opt. Lett. 33, 1026-1028 (2008); and Korlach et al. Proc. Natl. Acad. Sci. USA 105, 1176-1181 (2008), the disclosures of which are incorporated herein by reference in their entireties.

A filter assembly 128 of the optics system 102 can be configured to filter the fluorescent emissions from the fluorescent labels in the flowcell 114. The filter assembly 128 can include a plurality of optical filters for the user to select from, depending on the particular fluorophores used in a sequencing reaction. In one alternate embodiment, the computer system 106 may automatically determine which optical filters are to be used for a sequencing reaction, e.g., by scanning labels and/or barcodes attached to a sample vial and determining the particular fluorophores to be used in a sequencing reaction based on the labels and/or barcodes, or by retrieving information stored in the memory relating to previous sequencing reactions, and then control the filter assembly 128 to select and use the desired optical filters. More than one filter can be used at a time. Each filter can be a longpass filter, a shortpass filter, a bandstop filter, or a bandpass filter, depending on the types of fluorescent molecules being used in the system. For example, the user can select a first filter and a second filter. The first filter can be a bandpass filter selected to match the peak of the emission spectrum of a first fluorescent label. The second filter can be a bandpass filter selected to match the peak of the emission spectrum of a second fluorescent label. The gap between the transmission windows of the two bandpass filters can be, for example, at least 5, 10, 20, 30, 40, 50, 60, 70, 80, 90, 100, 200, 300, 400, 500 nm, or a number or a range between any two of these values, apart. The center of the transmission window of the first bandpass filter and the center of the transmission window of the second bandpass filter can be apart from each other, for example, ranging from 10 nm to 100 nm. The center of the transmission window of the first bandpass filter and the center of the transmission window of the second bandpass filter can be, or be about, 10, 11, 12, 13, 14, 15, 16, 17, 18, 19, 20, 30, 40, 50, 60, 70, 80, 90, 100, 200, 300, 400, 500, 600, 700, 800, 900, 1000 nm, or a number or a range between any two of these values, apart.

In some embodiments, the detector 126 includes one sub-detector while the filters of the filter assembly 128 may be mechanically switched or rotated in front of the sub-detector, such that differently filtered images can be taken by the sub-detector sequentially. In some embodiments, the detector 126 includes one sub-detector and the filter assembly 128 may include at least one layer of switchable material which has a light transmittance that is variable upon application of a stimulus, where the stimulus may be light, electricity, temperature, or any combination thereof. As a result, the filter assembly 128 can provide a plurality of optical filters such that differently filtered images can be taken by the sub-detector sequentially. In some embodiments, the detector 126 includes one sub-detector and the filter assembly 128 may include one or more switchable filters base on the micro-electromechanical system technology, such that differently filtered images can be taken by the sub-detector sequentially.

In some embodiments, the detector 126 can includes two or more sub-detectors, for example a first detector coupled with a first filter and a second detector coupled with a second filter, and the optics system 102 may include two or more dichroic mirrors/beamsplitters configured to split the fluorescent emissions. After splitting the fluorescent emissions with the dichroic mirrors, the detector 126 can take two differently filtered images simultaneously (or close in time) using the two sub-detectors coupled with two different filters, for example. In some embodiments, the detector 126 can includes two or more sub-detectors stacked along the incoming direction of the fluorescent emissions. Different wavelengths of the fluorescent emissions may differentially decay or be differentially absorbed along the incoming direction, such that sub-detectors at different positions along the incoming direction can be configured to take differently filtered images simultaneously (or close in time).

In use, a sample having a polynucleotide to be sequenced may be loaded into the flowcell 114 and placed in the mounting stage 116. The computer system 106 may then activate the fluidics system 104 to begin a sequencing cycle. During sequencing reactions, the computer system 106 may instruct the fluidics system 104, through the communication interface 108b, to supply reagents, for example labeled nucleotide analogs, to the flowcell 114. Through the communication interface 108a and the computer interface 110, the computer system 106 may control the light source 120 of the optics system 102 to generate light at around a predetermined wavelength and excite nucleotide analogs incorporated into growing primers hybridized to the polynucleotide being sequenced, for example. The computer system 106 may control the detector 126 of the optics system 102 to capture images of the diffraction-limited spots of DNA clusters having the fluorescently labeled nucleotide analogs. The computer system 106 can receive the fluorescent images from the detector 126 and process the fluorescent images received to determine the nucleotide sequence of the polynucleotide being sequenced.

In FIG. 1B, an example of an imaging system 10000 to be used in the disclosed sequencing technology is illustrated. For example, the imaging system 10000 may be used in the example sequencing system 100 illustrated in FIG. 1A. The imaging system 10000 may include a light source 11000 that can provide light to excite fluorophores at targeted points on a sample. The light source 11000 can include one or more lasers, light-emitting diodes, or other optical sources, such that the light source 11000 can provide a variety of wavelengths of light. In some embodiments, the light source 11000 can be configured to selectively provide light with a predetermined range of wavelengths that are tuned to the set of fluorophores being used. In some embodiments, the light source 11000 can be configured to output light at an optical frequency corresponding to a wavelength in a predefined range of wavelengths of light. In some embodiments, a user of the disclosed sequencing systems may choose a specific optical frequency to be output from the light source 11000, depending on the particular fluorophores used in a sequencing reaction.

The imaging system 10000 may include an optical path 12000 from the light source 11000 to the sample 13000, e.g., a microfluidic device including one or more flow chambers where one or more sequencing reactions occur. In some embodiments, the optical path 12000 can include a combination of one or more of mirrors, lenses, prisms, quarter wave plates, half wave plates, polarizers, filters, dichroic mirrors, beam splitters, beam combiners, objective lenses, wide field optics configured to spread light from a light source over a relatively large region of a sample, etc. The optical path 12000 can be configured to direct light from the light source 11000 to the sample 13000. In addition, the optical path 12000 may include optical components which can be configured to direct light emitted from the sample 13000 to an integration detection system 15000. In some embodiments, a portion of the optical elements that are used to direct light from the light source 11000 to the sample 13000 are also used to direct light from the sample 13000 to the integration detection system 15000. Further examples of optical paths and optical systems may be found in U.S. Pat. Nos. 7,589,315, 8,951,781, or 9,193,996, each of which is incorporated by reference herein in its entirety.

The imaging system 10000 may include a scanning system 14000 to effectively move light relative to the sample 13000 to scan the sample to generate an image. In some embodiments, the scanning system 14000 can be implemented within the optical path 12000. For example, the scanning system 14000 can include one or more scanning mirrors that move relative to one another within the optical path 12000 to effectively move the light from the light source 11000 across the sample. In some embodiments, the scanning system 14000 can be implemented as a mechanical system that physically moves the sample 13000 so that the sample moves relative to the light from the light source 11000. In some embodiment, the scanning system 14000 can be a combination of optical components in the optical path 12000 and a mechanical system for physically moving the sample 13000 so that the light from the light source 11000 and the sample 13000 move relative to one another.

The imaging system 10000 may include an integration detection system 15000 that includes one or more light detectors as well as associated electronic circuitry, processors, data storage, memory, and the like to acquire and process image data of the sample 13000. In some embodiments, the integration detection system 15000 can include photomultiplier tubes, avalanche photodiodes, image sensors (e.g., CCDs, CMOS sensors, etc.), and the like. In some embodiments, the light detectors of the integration detection system 15000 can include components to amplify light signals and may be sensitive to single photons. In some embodiments, the light detectors of the integration detection system 15000 can have a plurality of channels or pixels. The integration detection system 15000 can acquire one or more images based on the light detected from the sample 13000.

In some embodiments, the optical path 12000 may include an array generator 12100 that can generate a plurality of light exposure regions on the sample 13000. In some embodiments, the array generator 12100 can generate a certain light exposure pattern on the sample 13000. These light exposure regions can be scanned over the sample 13000 using the scanning system 14000 to selectively illuminate areas of the sample 13000 for imaging. The integration detection system 15000 can integrate signals corresponding to particular points on the sample 13000 as the plurality of light exposure regions are scanned over the sample 13000. For example, for an individual point on the sample 13000, the integration detection system 1500 can selectively aggregate detected signals corresponding to the individual point where the individual point is illuminated at different times by different light exposure regions. In some embodiments, the combination of the array generator 12100 and the integration detection system 15000 can detect light simultaneously, or near-simultaneously, from a plurality of points on the sample 13000. In some embodiments, the combination of the array generator 12100 and the integration detection system 15000 can integrate the detected light from a plurality of points on the sample over time.

In some embodiments, a plurality of sequencing reactions may be run parallelly in a plurality of flow chambers of the sample 13000. For example, a plurality of sequencing reactions may be performed for a plurality of biological specimen. In some embodiments, the plurality of sequencing reactions may use different sets of fluorophores. In some embodiments, the light source 11000, the array generator 12100, and the scanning system 14000 can be configured to selectively illuminate different areas of the sample 13000 with different optical frequencies of light, depending on the different sets of fluorophores used for the sequencing reactions occurring in different areas of the sample 13000.

In FIG. 1C, another example of an imaging system 1500 to be used in the disclosed sequencing technology is illustrated. For example, the imaging system 1500 may be used in the example sequencing system 100 illustrated in FIG. 1A. The imaging system 1500 may be used to image a flowcell 1600 having an upper layer 1671 and a lower layer 1673 that may be separated by a fluid filled channel 1675. In the configuration shown, the upper layer 1671 may be optically transparent and light from the imaging system 1500 may be focused to an area 1676 on the inner surface 1672 of the upper layer 1671. In an alternative configuration, light from the imaging system 1500 can be focused on the inner surface 1674 of the lower layer 1673. One or both of the surfaces can include array features which contain polynucleotides and sequencing reactions that are to be detected by the imaging system 1500.

The imaging system 1500 may include an objective 1501 that is configured to direct excitation light from a light source 1502 to the flowcell 1600 and to direct emission from the flowcell 1600 to a detector 1508. In the exemplary layout, excitation light from the light source 1502 passes through a lens 1505, then through a beam splitter 1506, and then through the objective 1501 on its way to the flowcell 1600. In some embodiments, the light source 1502 may include one or more lasers, light-emitting diodes, or any combination thereof. For example, the light source 1502 may include one laser 1503 and one light emitting diode 1504, which can provide light at different wavelengths or ranges of wavelengths to be selected by the user. The emission light from the flowcell 1600 may be captured by the objective 1501 and reflected by the beam splitter through the beam conditioning optics 1507 and to the detector 1508 (e.g. a CMOS sensor). The beam splitter 1506 may direct the emission light in a direction that is orthogonal to the path of the excitation light. The position of the objective 1501 can be moved in the z dimension to alter the focus of the excitation light on the flowcell 1600. The imaging system 1500 can be moved back and forth in the y direction to capture images of several areas of the flowcell 1600.

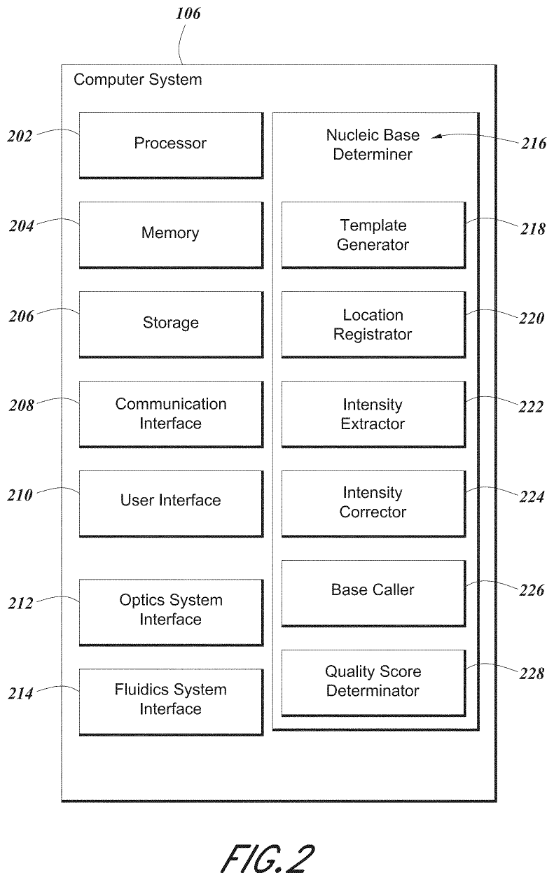

The computer system 106 of the example sequencing system 100 illustrated in FIG. 1A can be configured to control the optics system 102 and the fluidics system 104. While many configurations are possible for the computer system 106, one embodiment is illustrated in FIG. 2. As shown in FIG. 2, the computer system 106 can include a processor 202 that is in electrical communication with a memory 204, a storage 206, and a communication interface 208.

The processor 202 can be configured to execute instructions that cause the fluidics system 104 to supply reagents to the flowcell 114 during sequencing reactions. The processor 202 can execute instructions that control the light source 120 of the optics system 102 to generate light at around a predetermined wavelength. The processor 202 can execute instructions that control the detector 126 of the optics system 102 and receive data from the detector 126. The processor 202 can execute instructions to process data, for example fluorescent images, received from the detector 126 and to determine the nucleotide sequences of polynucleotides based on the data received form the detector 126.

The memory 204 can be configured to store instructions for configuring the processor 202 to perform the functions of the computer system 106 when the sequencing system 100 is powered on. When the sequencing system 100 is powered off, the storage 206 can store the instructions for configuring the processor 202 to perform the functions of the computer system 106. The communication interface 208 can be configured to facilitate the communications between the computer system 106, the optics system 102, and the fluidics system 104.

The computer system 106 can include a user interface 210 configured to communicate with a display device (not shown) for displaying the sequencing results of the single sequencing system 100. The user interface 210 can be configured to receive inputs from users of the sequencing system 100. An optics system interface 212 and a fluidics system interface 214 of the computer system 106 can be configured to control the optics system 102 and the fluidics system 104 through the communication links 108a and 108b illustrated in FIG. 1A. For example, the optics system interface 212 can communicate with the computer interface 110 of the optics system 102 through the communication link 108a.

The computer system 106 can include a nucleic base determiner 216 configured to determine the nucleotide sequence of polynucleotides using the data received from the detector 126. The nucleic base determiner 216 can include one or more of: a template generator 218, a location registrator 220, an intensity extractor 222, an intensity corrector 224, a base caller 226, and a quality score determiner 228. The template generator 218 can be configured to generate a template of the locations of polynucleotide clusters in the flowcell 114 using the fluorescent images captured by the detector 126. The location registrator 220 can be configured to register the locations of polynucleotide clusters in the flowcell 114 in the fluorescent images captured by the detector 126 based on the location template generated by the template generator 218. The intensity extractor 222 can be configured to extract intensities of the fluorescent emissions from the fluorescent images to generate extracted intensities. The intensity corrector 224 can be configured to reduce or eliminate the cross-talk between fluorescent labels in different optical channels by, for example, color correcting the extracted intensities to generate corrected intensities. In some embodiments, the intensity corrector 224 can phase correct or prephase correct extracted intensities. The base caller 226 can be configured to determine the nucleobases of a polynucleotide from the corrected intensities. The bases of a polynucleotide determined by the base caller 226 can be associated with quality scores determined by the quality score determiner 228. Further details of the computations that can be performed by the nucleic base determiner may be found in U.S. Patent Application Publication Numbers 2020/0080142 and 2012/0020537, each of which is incorporated by reference herein in its entirety.

### Sequencing With Single Excitation, Three-Label, Two-Optical Channel

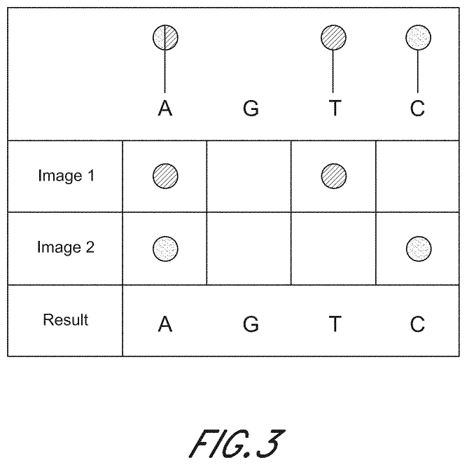

The disclosed technology may use a sequencing-by-synthesis process. During each sequencing cycle, four types of nucleotide analogs can be added and incorporated onto the growing primer-polynucleotides. The four types of nucleotide analogs can have different modifications. For example, as shown in FIG. 3, the first type of nucleotide can be an analog of deoxythymidine triphosphate (dTTP) conjugated with a first type of fluorescent label via a linker. The second type of nucleotide can be an analog of deoxycytidine triphosphate (dCTP) conjugated with a second type fluorescent label via a linker. The third type of nucleotide can be an analog of deoxyadenosine triphosphate (dATP) conjugated with a third type of fluorescent label via a linker. The fourth type of nucleotide can be an analog of deoxyguanosine triphosphate (dGTP) which is not conjugated with any fluorescent label. After the incorporation of nucleotide analogs, any unincorporated nucleotide analogs can be washed and removed.

The first fluorescent label may have an emission spectrum that can be captured in a first image taken in a first optical channel. The second fluorescent label may have an emission spectrum that can be captured in a second image taken in a second optical channel which is distinct from the first optical channel. The third fluorescent dye may have an emission spectrum that can be captured in both the first and second optical channels. In some embodiments, coupling of the dyes to nucleotides may not result in significant changes to their absorption or emission spectra. As a result, in the example shown in FIG. 3, dATP can be identified as showing in both the first and second images. dGTP may not show in either images. dTTP can be identified as showing only in the first image. dCTP can be identified as showing only in the second image.

The example nucleotides shown in FIG. 3 may be fully functionalized nucleotides. The linkers located between the nucleotide base and the fluorescent molecule may include one or more cleavage groups. Prior to the subsequent sequencing cycle, the fluorescent labels can be removed from the nucleotide analogs by cleavage of the linker. For example, a linker attaching a fluorescent label to a nucleotide analog can include an azide and/or an alkoxy group, for example on the same carbon, such that the linker may be cleaved after each incorporation cycle by a phosphine reagent, thereby releasing the fluorescent label. The nucleotide triphosphates can be reversibly blocked at the 3′ position so that sequencing is controlled, and no more than a single nucleotide analog can be added onto each extending primer-polynucleotide in each cycle. For example, the 3′ ribose position of a nucleotide analog can include both alkoxy and azido functionalities which can be removable by cleavage with a phosphine reagent, thereby creating a nucleotide that can be further extended. Prior to the subsequent sequencing cycle, the reversible 3′ blocks can be removed so that another nucleotide analog can be added onto each extending primer-polynucleotide.

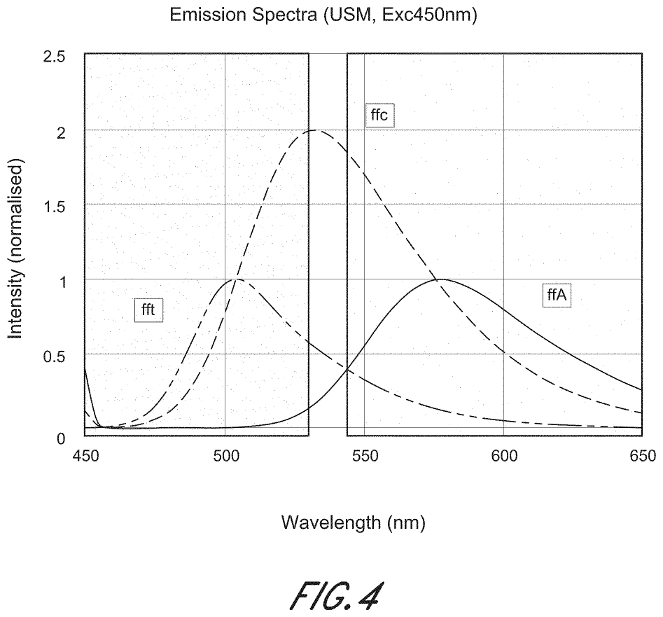

FIG. 4 shows example emission spectra of a collection of fully-functionalized nucleotides which can be used in embodiments of a single excitation, three-label, two-optical channel sequencing method. In this example, the fully-functionalized dTTP (ffT) is labeled with a first dye which has an emission spectrum shown as the curve having a peak at about 500 nm, when excited by a 450 nm light source. The fully-functionalized dATP (ffA) is labeled with a second dye which has an emission spectrum shown as the curve having a peak at about 575 nm, when excited by a 450 nm light source. dGTP is not labeled with any fluorescent dyes in this example. The fully-functionalized dCTP (ffC) is labeled with a third dye which has a relatively broad emission spectrum shown as the curve having a wide peak at about 535 nm, when excited by a 450 nm light source. In some embodiments, the emission spectrum of the third dye emits photons across a wider range of wavelengths as compared to the emission spectrum of the first dye or the second dye. In alternative embodiments, the third dye having the wider emission spectrum may be used in a two-excitation, two-optical channel sequencing method, e.g., a method where both a laser producing light in the blue range and a laser producing light in the green range are used to excite the dyes.

In FIG. 4, the first optical channel is represented by the window which spans from about 450 nm to about 530 nm. The second optical channel is represented by the window which spans from about 545 nm to about 650 nm. Therefore, when excited by a 450 nm light source, ffT emissions will result in a peak signal (e.g., power) in the first optical channel, and will only result in a small or negligible signal in the second optical channel. ffA emission will result in a peak signal in the second optical channel, and will only result in a small or negligible signal in the first optical channel. ffC emission will result in relatively large signals in both windows corresponding to the first optical channel and the second optical channel, since the emission spectrum of the third dye has a wider range of emission wavelengths as compared to the emission spectrum of the first dye or the second dye. Because dGTP is not conjugated to any fluorescent label, it will not fluoresce and will not be detected in the first optical channel or the second optical channel. In some embodiments, the third dye on ffC is chosen to be brighter than the other two dyes. As a result, the magnitude of the ffC signal in the first optical channel will be comparable to the magnitude of the ffT signal in the first optical channel, and the magnitude of the ffC signal in the second optical channel will be comparable to the magnitude of the ffA signal in the second optical channel.

In some embodiments, one of the three fluorescent dyes can be a normal Stokes shift dye. As used herein, a normal Stokes shift dye refers to a dye having a Stokes shift between 55 to 95 nm, or including Stokes shifts of about 55, 60, 70, 80, 90, 95 nm, or any value therebetween. One of the three fluorescent dyes can be a short Stokes shift dye. As used herein, a short Stokes shift dye refers to a dye having a Stokes shift of about 5, 10, 20, 30, 40, 50 nm, or any value therebetween. One of the three fluorescent dyes can be a long Stokes shift dye. As used herein, a long Stokes shift dye refers to a dye having a Stokes shift of about 100, 110, 120, 130, 140, 150, 160, 170, 180, 190, 200 nm, or any value therebetween.

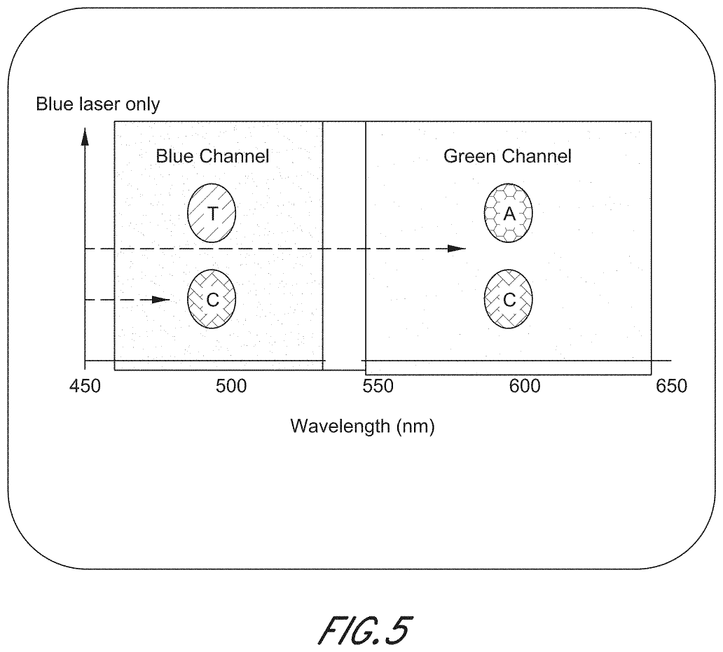

FIG. 5 schematically illustrates an example of single excitation, two-optical channel detection of fully-functionalized nucleotides labeled according to the example shown in FIG. 4. As shown in FIG. 5, a single light source, such as a “blue” laser, can excite the three fluorescent labels at a predetermined wavelength, such as 450 nm. In various embodiments, the output optical frequency of the single light source may or may not be tunable. Detection of the fluorescent labels can include capturing the fluorescent emissions in two distinct optical channels. For example, ffT and ffC are captured in a first image taken in the “blue” channel represented by the window which span from about 460 nm to about 530 nm. ffA and ffC are captured in a second image taken in the “green” channel represented by the window which span from about 550 nm to about 650 nm. In some embodiments, the fluorescent images can be stored for later processing offline. In some embodiments, the fluorescent images can be processed to determine the sequence of the growing primer-polynucleotides in each cluster in real time.

In some embodiments, the disclosed system may be used for identifying a nucleotide in a nucleic acid sequence bound to a substrate. In some embodiments, the disclosed system may include: a first detector configured to detect a first range of wavelengths of light; a second detector configured to detect a second range of wavelengths of light; a light source comprising a laser or a light-emitting diode which outputs light at an optical frequency; and a processor. The processor may be configured to: generate light at the optical frequency to stimulate an emission from the nucleic acid sequence on the substrate; and identify a nucleotide in the nucleic acid sequence based on whether the emission is received by the first detector, the second detector, both the first and second detectors, or neither the first nor second detector. In some embodiments, the first range of wavelengths and the second range of wavelengths do not overlap.

In some embodiments, the optical frequency corresponds to a wavelength in a predefined range of wavelengths of light, wherein the predefined range comprises at least one wavelength that is shorter than all of the wavelengths in the first range and in the second range. In some embodiments, the predefined range comprises 405 nm-460 nm. In some embodiments, the optical frequency corresponds to a wavelength in a predefined range of wavelengths of light, wherein the predefined range comprises at least one wavelength that is longer than some of the wavelengths in the first range or in the second range.

In some embodiments, the disclosed system may further include: a first nucleotide coupled to a first fluorescent label; a second nucleotide coupled to a second fluorescent label; a third nucleotide coupled to a third fluorescent label; and a fourth nucleotide coupled to no fluorescent label. The light source may be configured to: excite the first fluorescent label to emit light to be detectable by the first detector; excite the second fluorescent label to emit light to be detectable by the second detector; and excite the third fluorescent label to emit light to be detectable by both the first and second detectors. In some embodiments, the light source is configured to excite the fluorescent labels by two-photon absorption processes.

In some embodiments, a processor may be configured to: identify a nucleotide in the nucleic acid sequence based on an emission signal intensity received by the first detector and the second detector. The first fluorescent label may be identified as having a larger signal intensity received by the first detector compared to that received by the second detector. The second fluorescent label may be identified as having a larger signal intensity received by the second detector compared to that received by the first detector. The third fluorescent label may be identified as having a comparable signal intensity received by the first detector compared to that received by the second detector. The fourth fluorescent label may be identified as having a low signal intensity (e.g., substantially close to background level) received by the first detector and by the second detector.

In some embodiments, the emission spectrum of the third fluorescent label has a wider range of emission wavelengths as compared to the emission spectrum of the first fluorescent label or the second fluorescent label. In some embodiments, the third fluorescent label is chosen to have a greater intensity of emission (be brighter) than the first or second fluorescent labels.

In some embodiments, the first fluorescent label has a Stokes shift between 20 nm-50 nm, the second fluorescent label has a Stokes shift between 100 nm-130 nm, and the third fluorescent label has a Stokes shift between 60 nm-90 nm. In some embodiments, the first fluorescent label is not detectable by the second detector, and wherein the second fluorescent label is not detectable by the first detector. In some embodiments, the first fluorescent label is also detectable by the second detector, or wherein the second fluorescent label is also detectable by the first detector.

In some embodiments, the fluorescent labels are selected from the group consisting of polymethine derivatives, coumarin derivatives, benzopyran derivatives, chromenoquinoline derivatives, compounds containing bis-boron heterocycles such as BOPPY and BOPYPY. In some embodiments, the fluorescent labels are selected from the group consisting of:

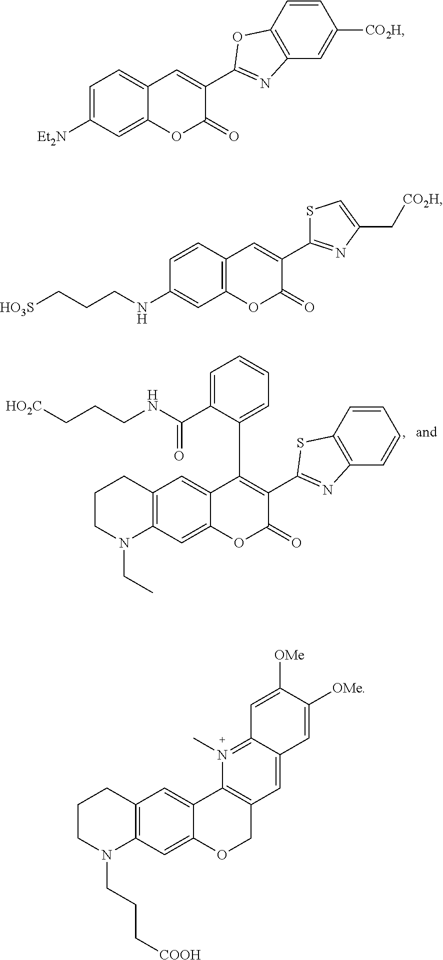

In some embodiments, the disclosed system may further include an additional first nucleotide coupled to no fluorescent label. The population or concentration of the additional first nucleotide coupled to no fluorescent label may be about 1, 5, 10, 15, 20, 25, 30, 35, 40, 45, 50%, or any value therebetween, of the total population or concentration of the first nucleotide. In some embodiments, the disclosed system may further include an additional first nucleotide coupled to an alternative fluorescent label, wherein the alternative fluorescent label cannot be excited by the light source to emit light to be detectable by the first detector. The population or concentration of the additional first nucleotide coupled to the alternative fluorescent label may be about 1, 5, 10, 15, 20, 25, 30, 35, 40, 45, 50%, or any value therebetween, of the total population or concentration of the first nucleotide. In some embodiments, the alternative fluorescent label and the first fluorescent label have different fluorescence emission spectra. In some embodiments, the disclosed system may further include an additional first nucleotide coupled to an alternate fluorescent label, wherein the alternate fluorescent label can be excited by the light source to emit light to be detectable by the first detector, and wherein the alternate fluorescent label emits dimmer light as compared to the first fluorescent label. The population or concentration of the additional first nucleotide coupled to the alternate fluorescent label may be about 1, 5, 10, 15, 20, 25, 30, 35, 40, 45, 50%, or any value therebetween, of the total population or concentration of the first nucleotide. In some embodiments, the alternate fluorescent label and the first fluorescent label have different fluorescence emission spectra.

In some embodiments, the alternative fluorescent label may be a fluorescent dye that can be excited by a “green” laser, for example, a light source having a wavelength between about 490 nm to 550 nm, e.g., about 532 nm. Non-limiting examples of the alternative fluorescent labels are disclosed in U.S. Pat. No. 10,982,261, which is incorporated by reference in its entirety. In one example, the alternative fluorescent label has the following structure:

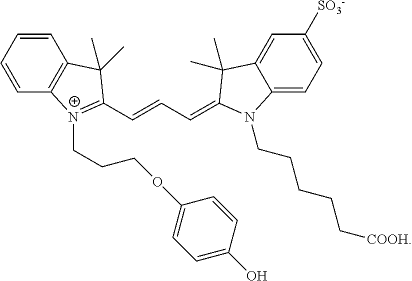

Other alternative fluorescent labels include those that can be excited by a “red” laser, for example, a light source having a wavelength between about 630 nm to about 700 nm, e.g., about 660 nm.

In some embodiments, the disclosed system may further include an additional second nucleotide coupled to no fluorescent label. The population or concentration of the additional second nucleotide coupled to no fluorescent label may be about 1, 5, 10, 15, 20, 25, 30, 35, 40, 45, 50%, or any value therebetween, of the total population or concentration of the second nucleotide. In some embodiments, the disclosed system may further include an additional second nucleotide coupled to an alternative fluorescent label, wherein the alternative fluorescent label cannot be excited by the light source to emit light to be detectable by the second detector. The population or concentration of the additional second nucleotide coupled to the alternative fluorescent label may be about 1, 5, 10, 15, 20, 25, 30, 35, 40, 45, 50%, or any value therebetween, of the total population or concentration of the second nucleotide. In some embodiments, the alternative fluorescent label and the second fluorescent label have different fluorescence emission spectra. In some embodiments, the disclosed system may further include an additional second nucleotide coupled to an alternate fluorescent label, wherein the alternate fluorescent label can be excited by the light source to emit light to be detectable by the second detector, and wherein the alternate fluorescent label emits dimmer light as compared to the second fluorescent label. The population or concentration of the additional second nucleotide coupled to the alternate fluorescent label may be about 1, 5, 10, 15, 20, 25, 30, 35, 40, 45, 50%, or any value therebetween, of the total population or concentration of the second nucleotide. In some embodiments, the alternate fluorescent label and the second fluorescent label have different fluorescence emission spectra.

In some embodiments, the disclosed system may further include an additional third nucleotide coupled to no fluorescent label. The population or concentration of the additional third nucleotide coupled to no fluorescent label may be about 1, 5, 10, 15, 20, 25, 30, 35, 40, 45, 50%, or any value therebetween, of the total population or concentration of the third nucleotide. In some embodiments, the disclosed system may further include an additional third nucleotide coupled to an alternative fluorescent label, wherein the alternative fluorescent label cannot be excited by the light source to emit light to be detectable by the first or second detectors. The population or concentration of the additional third nucleotide coupled to the alternative fluorescent label may be about 1, 5, 10, 15, 20, 25, 30, 35, 40, 45, 50%, or any value therebetween, of the total population or concentration of the third nucleotide. In some embodiments, the alternative fluorescent label and the third fluorescent label have different fluorescence emission spectra. In some embodiments, the disclosed system may further include an additional third nucleotide coupled to an alternate fluorescent label, wherein the alternate fluorescent label can be excited by the light source to emit light to be detectable by the first and second detectors, and wherein the alternate fluorescent label emits dimmer light as compared to the third fluorescent label. The population or concentration of the additional third nucleotide coupled to the alternate fluorescent label may be about 1, 5, 10, 15, 20, 25, 30, 35, 40, 45, 50%, or any value therebetween, of the total population or concentration of the third nucleotide. In some embodiments, the alternate fluorescent label and the third fluorescent label have different fluorescence emission spectra.

In some embodiments, the detectors may include complementary metal-oxide-semiconductor image sensors, charge-coupled device image sensors, photomultiplier tubes, photodiodes, or any combination thereof. In some embodiments, the disclosed system may further include one or more optical filter materials, one or more diffraction gratings, one or more light dispersing elements, or any combination thereof. In some embodiments, the disclosed system may further include a polymerase configured to replicate or transcribe a portion of the nucleic acid sequence by incorporating the nucleotides. In some embodiments, the substrate comprises a plurality of chemically functionalized regions, a plurality of cavities, a plurality of optical resonators, a plurality of optical waveguides, or any combination thereof. In some embodiments, the fluorescent label is attached to the nucleotide through a cleavable linker. In some further embodiments, the labeled nucleotide may have the fluorescent label attached to the C5 position of a pyrimidine base or the C7 position of a 7-deaza purine base, optionally through a cleavable linker moiety. For example, the nucleobase may be 7-deaza adenine and the dye is attached to the 7-deaza adenine at the C7 position, optionally through a cleavable linker. The nucleobase may be 7-deaza guanine and the dye is attached to the 7-deaza guanine at the C7 position, optionally through a cleavable linker. The nucleobase may be cytosine and the dye is attached to the cytosine at the C5 position, optionally through a cleavable linker. As another example, the nucleobase may be thymine or uracil and the dye is attached to the thymine or uracil at the C5 position, optionally through a cleavable linker. In some further embodiments, the cleavable linker may comprise similar or the same chemical moiety as the reversible terminator 3′ hydroxy blocking group such that the 3′ hydroxy blocking group and the cleavable linker may be removed under the same reaction condition or in a single chemical reaction. Non-limiting example of the cleavable linker include the LN3 linker, the sPA linker, and the AOL linker, each of which is exemplified below.

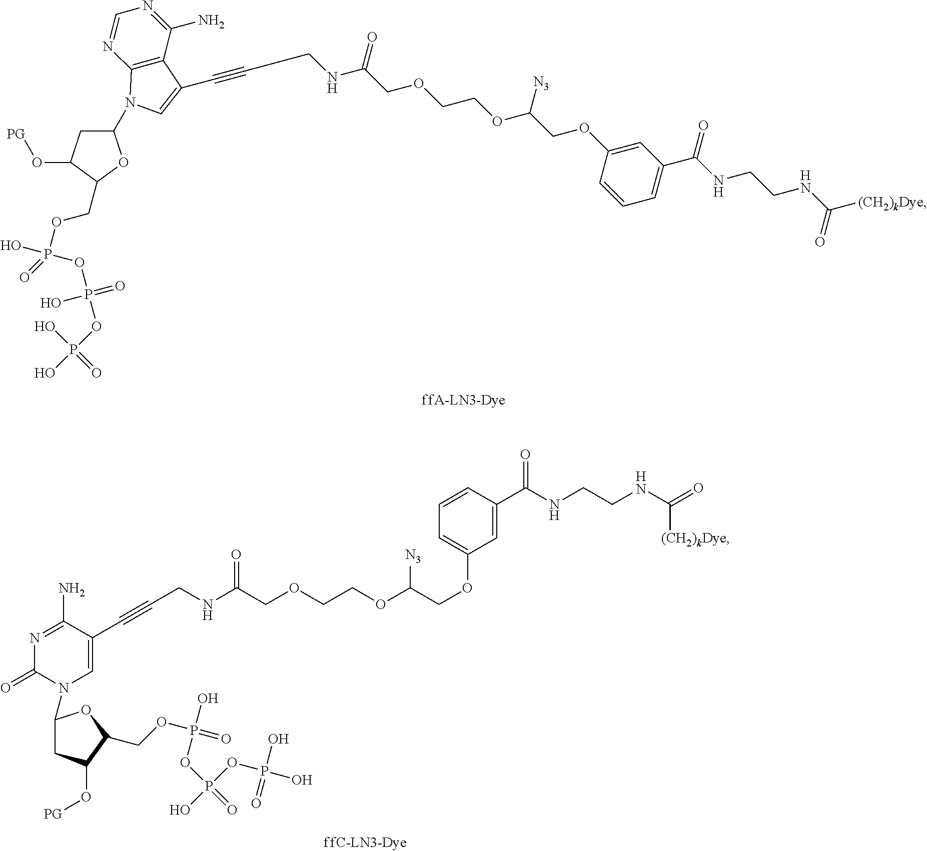

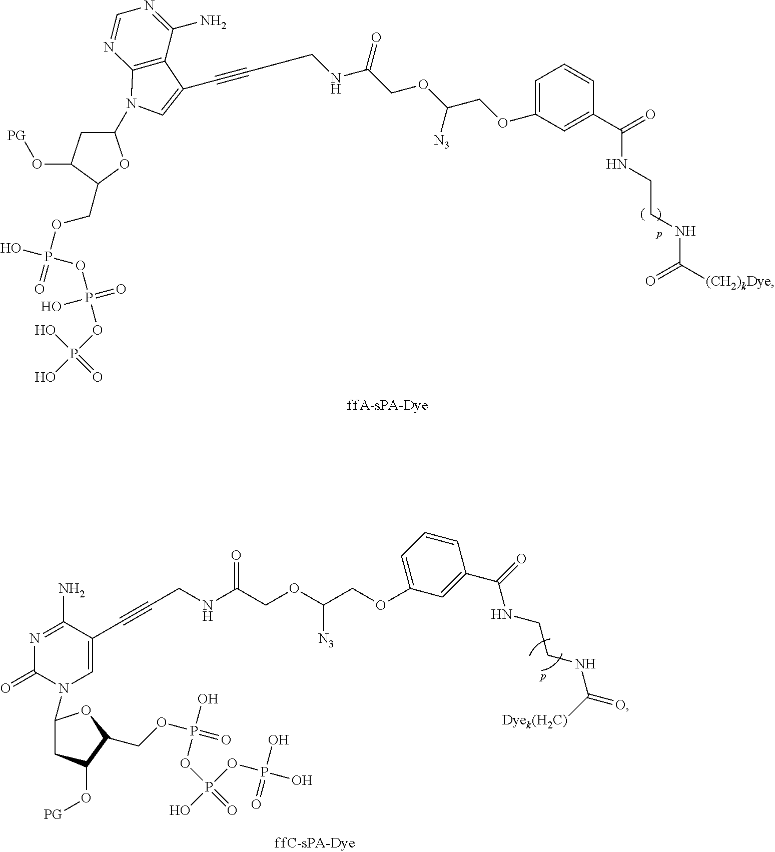

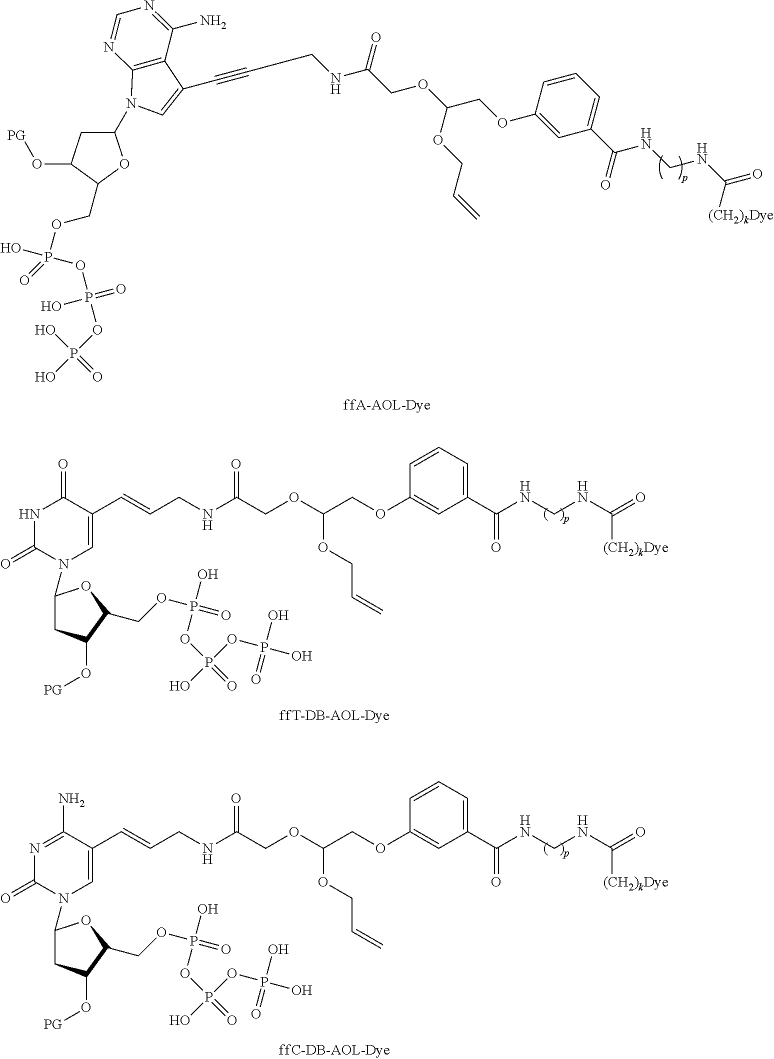

In some embodiments, the nucleotides are selected from the group consisting of an analog of dGTP, an analog of dTTP, an analog of dUTP, an analog of dCTP, and an analog of dATP. In some embodiments, the first nucleotide is a first reversibly blocked nucleotide triphosphate (rbNTP), the second nucleotide is a second rbNTP, the third nucleotide is a third rbNTP, and the fourth nucleotide is a fourth rbNTP, wherein each of the first nucleotide, second nucleotide, third nucleotide and fourth nucleotide is a different type of nucleotide from the other. In some embodiments, the four rbNTPs are selected from the group consisting of rbATP, rbTTP, rbUTP, rbCTP, and rbGTP. In some embodiments, each of the four rbNTPs includes a modified base and a reversible terminator 3′ blocking group. Non-limiting example of the 3′ blocking group include azidomethyl (*—CH₂N₃), substituted azidomethyl (e.g., *—CH(CHF₂)N₃ or *—CH(CH₂F)N₃) and *—CH₂—O—CH₂—CH═CH₂, where the asterisk * indicates the point attachment to the 3′ oxygen of the ribose or deoxyribose ring of the nucleotide.

In some embodiments, the disclosed single excitation, three-label, two-optical channel sequencing method may be implemented in an Illumina NextSeq 500®, NextSeq 550®, NextSeq 1000®, NextSeq 2000®, all NovaSeq®, or MiniSeq® system.

In some embodiments, dTTP is coupled to

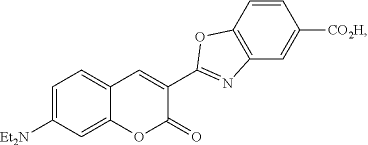

and the ffT has an emission maximum at about 499 nm when excited by light at about 450 nm to about 460 nm.

In some embodiments, dTTP is coupled to

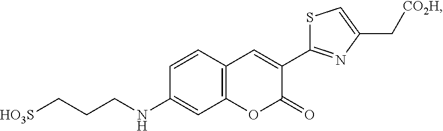 [SMILES: O=C(O)Cc1csc(-c2cc3ccc(NCCCS(=O)(=O)O)cc3oc2=O)n1]

and the ffT has an emission maximum at about 490 nm when excited by light at about 450 nm to about 460 nm.

In some embodiments, dCTP is coupled to

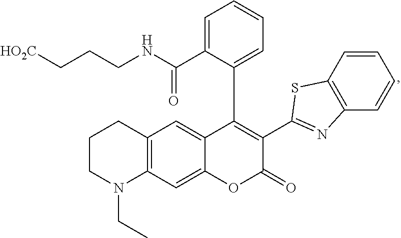 [SMILES: CCN1CCCc2cc3c(-c4ccccc4C(=O)NCCCC(=O)O)c(-c4nc5ccccc5s4)c(=O)oc3cc21]

and the ffC has an emission maximum at about 540 nm when excited by light at about 450 nm to about 460 nm.

In some embodiments, dATP is coupled to

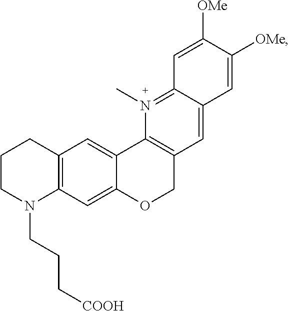

and the ffA has an emission maximum at about 580 nm when excited by light at about 450 nm to about 460 nm.

Further details about the dyes and the fully functionalized nucleotides can be found in U.S. Patent Application Publication Numbers 2018/0094140 and 2020/0277670, International Patent Application Publication Number 2017/051201, and U.S. Provisional Patent Application Nos. 63/057758 and 63/127061, the disclosures of which are incorporated herein by reference in their entireties.

### EXAMPLES

### Experiment 1

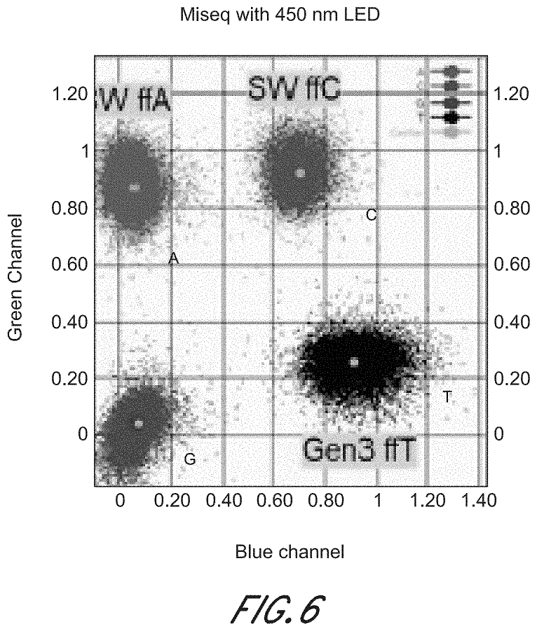

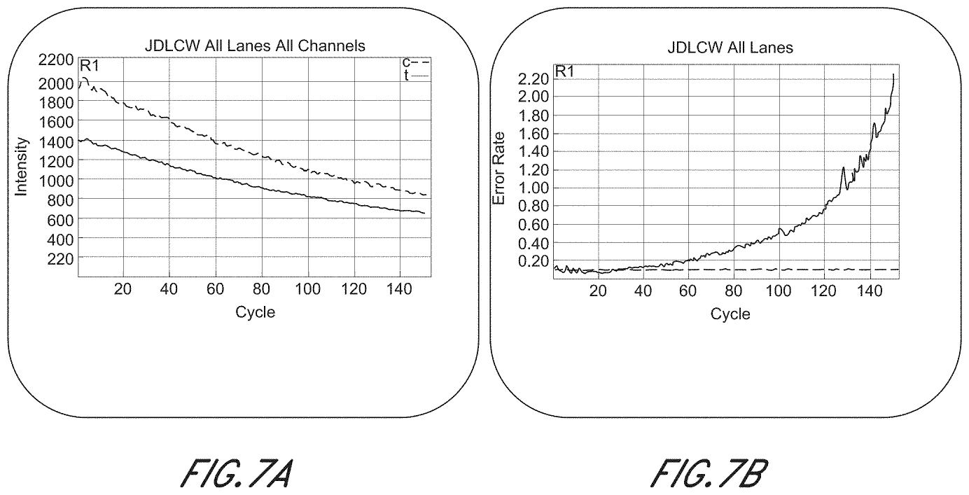

FIG. 6, FIG. 7A and FIG. 7B show results of a sequencing experiment performed according to the disclosed technology consistent with the examples illustrated in FIG. 4 and FIG. 5. A sequencing run was performed on an Illumina MiSeq system for about 150 sequencing cycles. A 450 nm LED was used as the excitation light source, and the exposure time was about 1000 ms for each image taken. In this experiment, dTTP was coupled to

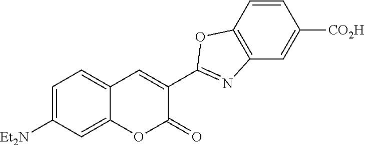

and the ffT had an emission maximum at about 499 nm, and an additional population of dTTP having no dye was used. dCTP was coupled to

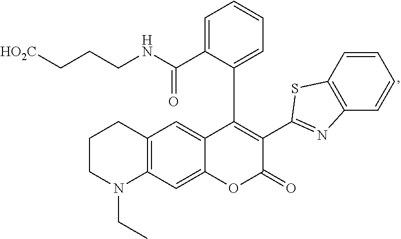 [SMILES: CCN1CCCc2cc3c(-c4ccccc4C(=O)NCCCC(=O)O)c(-c4nc5ccccc5s4)c(=O)oc3cc21]

and the ffC had an emission maximum at about 540 nm. dATP was coupled to

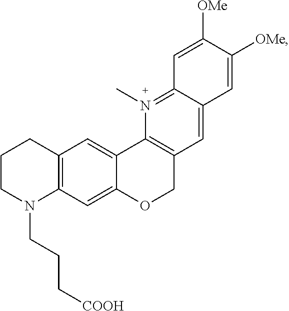

and the ffA had an emission maximum at about 580 nm. FIG. 6 shows a scatter plot of DNA cluster signals extracted from images of the sample flowcell across about 150 sequencing cycles. The horizontal axis represents signal intensity extracted from the image taken in a first “blue” optical channel, and the vertical axis represents signal intensity extracted from the image taken in a second “green” optical channel. The clusters which resulted in a large signal in the first “blue” optical channel and only a small signal in the second “green” optical channel were identified as having a T base, since ffT was labeled with the first dye. The clusters which resulted in a large signal in the second “green” optical channel and only a small signal in the first “blue” optical channel were identified as an A base, since ffA was labeled with the second dye. The clusters resulted in large enough signals in both the first optical channel and the second optical channel can be identified as a C base, since ffC was labeled with the third dye. The clusters resulted in minimal signal in either the first optical channel or the second optical channel can be identified as have a G base, since dGTP is not labeled with any dye.

The groups of clusters as shown in FIG. 6 were readily separable, thus base calling can be reliably achieved using this single light source, three dye sequencing process. Based on the same experimental data shown in FIG. 6, FIG. 7A shows, for clusters identified as the T base and for clusters identified as the C base, the DNA cluster signal intensity (averaged over clusters identified as having the same base in the sample flowcell) as the sequencing cycles progressed. As shown, the DNA cluster signal intensity decayed as the number of sequencing cycles increased, but the signal intensity was still high enough after about 150 sequencing cycles. FIG. 7B shows the base calling error rate (%) as the sequencing cycles progressed. As shown, the error rate increased as the number of sequencing cycles increased, but the error rate remained low enough (below 2.2%) after about 150 sequencing cycles.

### Experiment 2

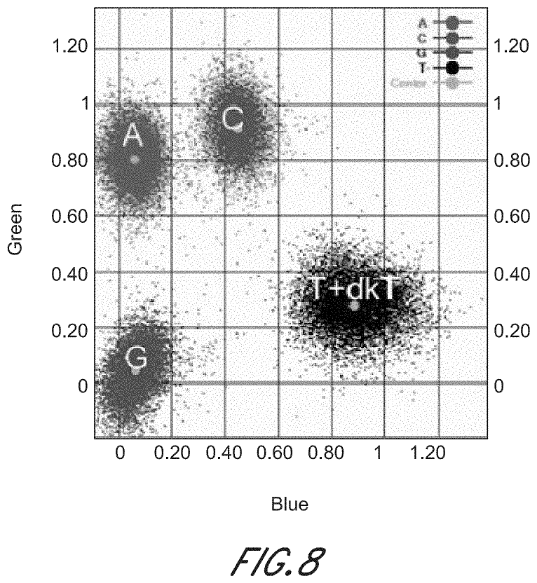

FIG. 8 shows a scatter plot similar to that described in connection with FIG. 6, but based on results of an additional sequencing experiment. The additional sequencing experiment used conditions similar to those described in connection with FIG. 6, but the dye used to label dTTP was

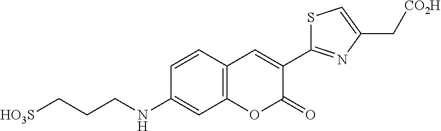 [SMILES: O=C(O)Cc1csc(-c2cc3ccc(NCCCS(=O)(=O)O)cc3oc2=O)n1]

and the ffT had an emission maximum at about 490 nm, and an additional population of dTTP having no dye was also used. The relative amount of dTTP having the first dye to dTTP having no dye was about 1:3. As shown in FIG. 8, using two populations of dTTP could tune the shapes of the cluster groups in the scatter plot and can affect the subsequent base calling performance.

### Experiment 3

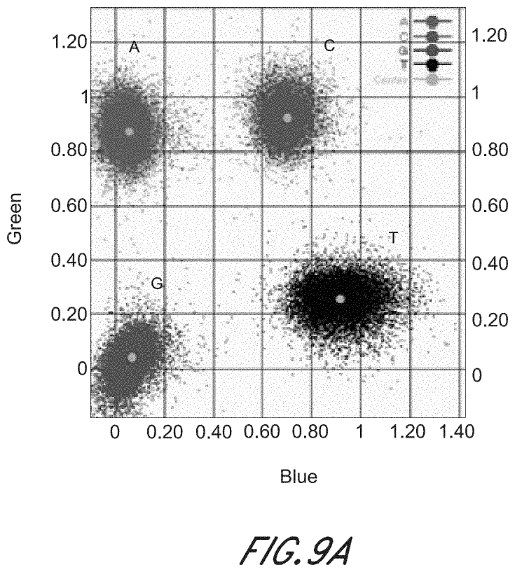

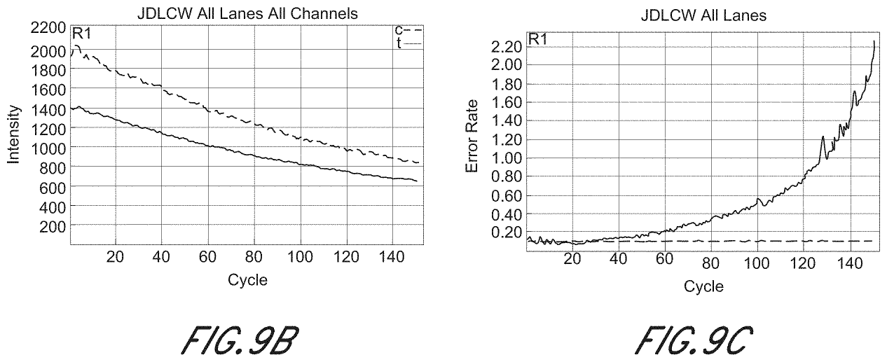

FIG. 9A shows a scatter plot similar to that described in connection with FIG. 8, but based on results of an alternative additional sequencing experiment. The alternative additional sequencing experiment used conditions similar to those described in connection with FIG. 8, but an alternative additional population of dTTP having a fourth dye,

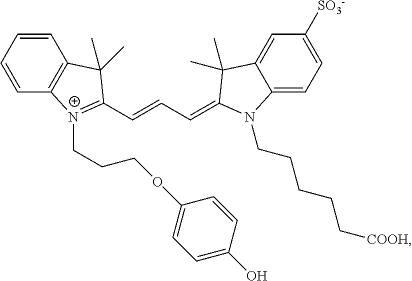

was used, in addition to the dTTP population labeled with

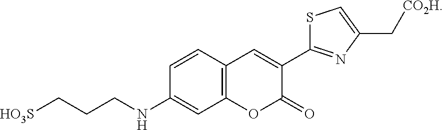 [SMILES: O=C(O)Cc1csc(-c2cc3ccc(NCCCS(=O)(=O)O)cc3oc2=O)n1]

The fourth dye was not excitable by the 450 nm LED. The relative amount of dTTP having the first dye to dTTP having the fourth dye was about 1.25:0.75. As shown in FIG. 9A, using the two populations of dTTP could further tune the shapes of the cluster groups in the scatter plot and can affect the subsequent base calling performance. Based on the same experimental data shown in FIG. 9A, FIG. 9B shows, for clusters identified as the T base and for clusters identified as the C base, the DNA cluster signal intensity (averaged over clusters identified as having the same base in the sample flowcell) as the sequencing cycles progressed. As shown, the DNA cluster signal intensity decayed as the number of sequencing cycles increased, but the signal intensity was still high enough after about 150 sequencing cycles. FIG. 9C shows the base calling error rate (%) as the sequencing cycles progressed. As shown, the error rate increased as the number of sequencing cycles increased, but the error rate remained low enough (below 2.2%) after about 150 sequencing cycles.

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originated in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydroshear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Standard protocols, e.g., protocols for sequencing using, for example, the Illumina platform, instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Sequencing Techniques

The disclosed sequencing systems and methods may be compatible with any sequencing techniques based on optical detection, for example, next-generation sequencing (NGS), fluorescent in situ sequencing (FISSEQ), and Massively Parallel Signature Sequencing (MPSS). In one embodiment, the disclosed systems and methods may be compatible with NGS technologies that allow multiple samples to be sequenced individually as genomic molecules (i.e., singleplex sequencing) or as pooled samples comprising indexed genomic molecules (e.g., multiplex sequencing) on a single sequencing run. These methods can generate up to several hundred million reads of DNA sequences.

The disclosed technology may implement sequencing reactions such as those incorporating sequencing-by-synthesis methods described in U.S. Patent Application Publication Numbers 2007/0166705, 2006/0188901, 2006/0240439, 2006/0281109, 2005/0100900, U.S. Pat. No. 7,057,026, PCT Application Publication Numbers WO 2005/065814, WO 2006/064199, and WO 2007/010251, the disclosures of which are incorporated herein by reference in their entireties. In some embodiments, the sequencers may implement sequencing-by-synthesis methods similar to those used in the HiSeq, MiSeq, or HiScanSQ systems from Illumina (San Diego, Calif.).

Alternatively, sequencing by ligation techniques may be used in the disclosed technology, such as described in U.S. Pat Nos. 6,969,488, 6,172,218, and 6,306,597, the disclosures of which are incorporated herein by reference in their entireties. Sequencing by ligation techniques use DNA ligase to incorporate oligonucleotides and identify the incorporation of such oligonucleotides.

The disclosed technology may be implemented in some sequencing techniques which are available commercially, such as the sequencing-by-hybridization platform from Affymetrix Inc. (Sunnyvale, Calif.) and the sequencing-by-synthesis platforms from 454 Life Sciences (Bradford, Conn.) and Helicos Biosciences (Cambridge, Mass.), the sequencing-by-ligation platform from Applied Biosystems (Foster City, Calif.), or the SMRT technology of Pacific Biosciences.

In one illustrative, but non-limiting, embodiment, the methods described herein comprise obtaining sequence information for the nucleic acids in a sample using Illumina's sequencing-by-synthesis and reversible terminator-based sequencing chemistry (e.g. as described in Bentley et al., Nature 6: 53-59 [2009]). Illumina's sequencing technology may include the attachment of fragmented genomic DNA to a planar, optically transparent surface on which oligonucleotide anchors are bound. For example, template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flowcell anchor oligos. Under limiting-dilution conditions, adapter-modified, single-stranded template DNA is added to the flowcell and immobilized by hybridization to the anchor oligos. Attached DNA fragments are extended and bridge amplified to create an ultra-high density sequencing flowcell with hundreds of millions of clusters, each containing about 1,000 copies of the same template. In one embodiment, the randomly fragmented genomic DNA is amplified using PCR before it is subjected to cluster amplification. Alternatively, an amplification-free (e.g., PCR free) genomic library preparation is used, and the randomly fragmented genomic DNA is enriched using the cluster amplification alone (Kozarewa et al., Nature Methods 6: 291-295 [2009]). The sequencing-by-synthesis reaction may employ reversible terminators with removable fluorescent dyes. Short sequence reads of about tens to a few hundred base pairs are aligned against a reference genome and unique mapping of the short sequence reads to the reference genome are identified. After completion of the first read, the templates can be regenerated in situ to enable a second read from the opposite end of the fragments. Thus, either single-end or paired end sequencing of the DNA fragments can be used. Detailed information about paired end sequencing can be found in U.S. Pat. No. 7601499 and US Patent Publication No. 2012/0,053,063, which are incorporated by reference.

In some embodiments, the sequencing by synthesis platform by Illumina involves clustering fragments. Clustering is a process in which each fragment molecule is isothermally amplified. In some embodiments, the fragment has two different adaptors attached to the two ends of the fragment, the adaptors allowing the fragment to hybridize with the two different oligos on the surface of a flowcell lane. The fragment further includes or is connected to two index sequences at two ends of the fragment, where index sequences provide labels to identify different samples in multiplex sequencing.

In some implementation, a flowcell for clustering in the Illumina platform is a glass slide with lanes. Each lane is a glass channel coated with a lawn of two types of oligos. Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification, a strand folds over, and a second adapter region on a second end of the strand hybridizes with the second type of oligos on the flowcell surface. A polymerase generates a complimentary strand, forming a double-stranded bridge molecule. This double-stranded molecule is denatured resulting in two single-stranded molecules tethered to the flowcell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters, or thousands to tens of thousands of millions of clusters, are sequenced in a massively parallel manner. At the completion of the first read, the read product is washed away.

In processes involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flowcell. An index 2 sequence is read in the same manner as index 1. Then an index 2 read product is washed off at the completion of the step.

After reading two indices, read 2 initiates by using polymerases to extend the second flowcell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

### Computing Systems

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6- diaminopurine, N6-ethano-2,6-diaminopurine, 5-methylcytosine, 5-(C3-C6)- alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The polymerase used is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, TIi DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants. “Encode” or “parse” are verbs referring to transferring from one format to another, and refers to transferring the genetic information of target template base sequence into an arrangement of reporters.

As used herein, the terms “well”, “cavity” and “chamber” are used synonymously, and refer to a discrete feature defined in the device that can contain a fluid (e.g., liquid, gel, gas). Examples of an array of the present device may have one or multiple wells. Further, it is to be understood that the cross-section of a well taken parallel to a surface of a substrate at least partially defining the well can be curved, square, polygonal, hyperbolic, conical, angular, etc.

A “light source” may be any device capable of emitting energy along the electromagnetic spectrum. A light source may be a source of visible light (VIS), ultraviolet light (UV) and/or infrared light (IR). “Visible light” (VIS) generally refers to the band of electro-magnetic radiation with a wavelength from about 400 nm to about 750 nm. “Ultraviolet (UV) light” generally refers to electromagnetic radiation with a wavelength shorter than that of visible light, or from about 10 nm to about 400 nm range. “Infrared light” or infrared radiation (IR) generally refers to electromagnetic radiation with a wavelength greater than the VIS range, or from about 750 nm to about 50,000 nm. A light source may also provide full spectrum light. Light sources may output light from a selected wavelength or a range of wavelengths. In some embodiments of the invention, the light source may be configured to provide light above or below a predetermined wavelength, or may provide light within a predetermined range. A light source may be used in combination with a filter, to selectively transmit or block light of a selected wavelength from the light source. A light source may be connected to a power source by one or more electrical connectors; an array of light sources may be connected to a power source in series or in parallel. A power source may be a battery, or a vehicle electrical system or a building electrical system. The light source may be connected to a power source via control electronics (control circuit); control electronics may comprise one or more switches. The one or more switches may be automated, or controlled by a sensor, timer or other input, or may be controlled by a user, or a combination thereof. For example, a user may operate a switch to turn on a UV light source; the light source may be applied on a constant basis until it is turned off, or it may be pulsed (repeated on/off cycles) until it is turned off. In some embodiments, the light source may be switched from a continuously-on state to a pulsed state, or vice versa. In some embodiments, the light source may be configured to be brightening or darkening over time.

For operation, the light source may be connected to a power source capable of providing sufficient power to illuminate the sample. Control electronics may be used to switch the power on or off based on input from a user or some other input, and can also be used to modulate the power to a suitable level (e.g. to control brightness of the output light). Control electronics may be configured to turn the light source on and off as desired. Control electronics may include a switch for manual, automatic, or semi-automatic operation of the light sources. The one or more switches may be, for example, a transistor, a relay or an electromechanical switch. In some embodiments, the control circuit may further comprise an AC-DC and/or a DC-DC converter for converting the voltage from the voltage source to an appropriate voltage for the light source. The control circuit may comprise a DC-DC regulator for regulation of the voltage. The control circuit may further comprise a timer and/or other circuitry elements for applying electric voltage to the optical filter for a fixed period of time following the receipt of input. A switch may be activated manually or automatically in response to predetermined conditions, or with a timer. For example, control electronics may process information such as user input, stored instructions, or the like.

One or more of a plurality of light sources may be provided. In some embodiments, each of the plurality of light sources may be the same. Alternatively, one or more of the light sources may vary. The light characteristics of the light emitted by the light sources may be the same or may vary. A plurality of light sources may or may not be independently controllable. One or more characteristic of the light source may or may not be controlled, including but not limited to whether the light source is on or off, brightness of light source, wavelength of light, intensity of light, angle of illumination, position of light source, or any combination thereof.

In some embodiments, light output from a light source may be from about 350 to about 750 nm, or any amount or range therebetween, for example from about 350 nm to about 360, 370, 380, 390, 400, 410, 420, 430 or about 450 nm, or any amount or range therebetween. In other embodiments, light from a light source may be from about 550 to about 700 nm, or any amount or range therebetween, for example from about 550 to about 560, 570, 580, 590, 600, 610, 620, 630, 640, 650, 660, 670, 680, 690 or about 700 nm, or any amount or range therebetween. In some embodiments, the wavelength of the light generated by the light source can vary, for example, ranging from 400 nm to 800 nm. In some embodiments, the wavelength of the light generated by the light source can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments, the wavelength of the light generated by the light source can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm. The light source may be capable of emitting electromagnetic waves in any spectrum. In some embodiments, the light source may have a wavelength falling between 10 nm and 100 μm. In some embodiments, the wavelength of light may fall between 100 nm to 5000 nm, 300 nm to 1000 nm, or 400 nm to 800 nm. In some embodiments, the wavelength of light may be less than, and/or equal to 10 nm, 100 nm, 200 nm, 300 nm, 400 nm, 500 nm, 600 nm, 700 nm, 800 nm, 900 nm, 1000 nm, 1100 nm, 1200 nm, 1300 nm, 1500 nm, 1750 nm, 2000 nm, 2500 nm, 3000 nm, 4000 nm, or 5000 nm.

In one example, a light source may be a light-emitting diode (LED) (e.g., gallium arsenide (GaAs) LED, aluminum gallium arsenide (AlGaAs) LED, gallium arsenide phosphide (GaAsP) LED, aluminum gallium indium phosphide (AlGaInP) LED, gallium(III) phosphide (GaP) LED, indium gallium nitride (InGaN)/gallium(III) nitride (GaN) LED, or aluminum gallium phosphide (AlGaP) LED). In another example, a light source can be a laser, for example a vertical cavity surface emitting laser (VCSEL) or other suitable light emitter such as an Indium-Gallium-Aluminum-Phosphide (InGaAIP) laser, a Gallium-Arsenic Phosphide/Gallium Phosphide (GaAsP/GaP) laser, or a Gallium-Aluminum-Arsenide/Gallium-Aluminum-Arsenide (GaAIAs/GaAs) laser. Other examples of light sources may include but are not limited to electron stimulated light sources (e.g., Cathodoluminescence, Electron Stimulated Luminescence (ESL light bulbs), Cathode ray tube (CRT monitor), Nixie tube), incandescent light sources (e.g., Carbon button lamp, Conventional incandescent light bulbs, Halogen lamps, Globar, Nernst lamp), electroluminescent (EL) light sources (e.g., Light-emitting diodes—Organic light-emitting diodes, Polymer light-emitting diodes, Solid-state lighting, LED lamp, Electroluminescent sheets Electroluminescent wires), gas discharge light sources (e.g., Fluorescent lamps, Inductive lighting, Hollow cathode lamp, Neon and argon lamps, Plasma lamps, Xenon flash lamps), or high-intensity discharge light sources (e.g., Carbon arc lamps, Ceramic discharge metal halide lamps, Hydrargyrum medium-arc iodide lamps, Mercury-vapor lamps, Metal halide lamps, Sodium vapor lamps, Xenon arc lamps). Alternatively, a light source may be a bioluminescent, chemiluminescent, phosphorescent, or fluorescent light source.

Optical filters may be tuned in terms of clarity or haze, translucency, transparency or opacity, light transmittance (LT), switching speed, durability, photostability, contrast ratio, state of light transmittance (e.g. dark state or light state). “Light transmittance” (LT) refers to the quantity of light that is transmitted or passes through an optical filter, or device or apparatus comprising same. LT may be expressed with reference to a change in light transmission and/or a particular type of light or wavelength of light (e.g. from about 10% visible light transmission (LT) to about 90% LT, or the like). LT may alternately be expressed as absorbance, and may optionally include reference to one or more wavelengths that are absorbed. According to some embodiments, an optical filter may be selected, or configured to have in one state, a LT of less than 80%, or less than 70%, or less than 60%, or less than 50%, or less than 40%, or less than 30%, or less than 20% or less than 10%, or any amount or range therebetween. According to some embodiments, an optical filter may be selected, or configured to have in another state, a LT of greater than 80%, or greater than 70%, or greater than 60%, or greater than 50%, or greater than 40%, or greater than 30%, or greater than 20% or greater than 10%, or any amount or range therebetween.

A filter can be a bandpass filter and can have peak transmittance of varying wavelength, ranging from 400 nm to 800 nm. In some embodiments, the peak transmittance can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments, the peak transmittance can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm. The width of the transmission window of a filter can vary, for example, ranging from 1 nm to 50 nm. In some embodiments, the width of the filter can be, or be about, 1, 2, 3, 4, 5, 6, 7, 8, 9, 10, 20, 30, 40, 50 nm, or a number or a range between any two of these values. In some embodiments, the width of the filter can be at least, or at most, 1, 2, 3, 4, 5, 6, 7, 8, 9, 10, 20, 30, 40, or 50 nm. A shortpass filter may be considered a special bandpass filter having the lower limit of the transmission window close to 0 nm. A longpass filter may be considered a special bandpass filter having the upper limit of the transmission window close to infinity. A bandstop filter may be defined as complementary to some bandpass filter.

Nucleosides and nucleotides may be labeled at sites on the sugar or nucleobase. A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. A linker group may be used to covalently attach a dye to the nucleoside or nucleotide.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

A nucleotide analog may be attached to or associated with a photo-detectable label via a linker to provide a detectable signal. In some embodiments, the photo-detectable label is a fluorescent compound, such as a small molecule fluorescent label. Fluorescent molecules (fluorophores) suitable as a fluorescent label include, but are not limited to: 1,5 IAEDANS; 1,8-ANS; 4-methylumbelliferone; 5-carboxy-2,7-dichlorofluorescein; 5-carboxyfluorescein (5-FAM); fluorescein amidite (FAM); 5-carboxynapthofluorescein; tetrachloro-6-carboxyfluorescein (TET); hexachloro-6-carboxyfluorescein (HEX); 2,7-dimethoxy-4,5-dichloro-6-carboxyfluorescein (JOE); VIC®; NED™; tetramethylrhodamine (TMR); 5-carboxytetramethylrhodamine (5-TAMRA); 5-HAT (Hydroxy Tryptamine); 5-hydroxy tryptamine (HAT); 5-ROX (carboxy-X-rhodamine); 6-carboxyrhodamine 6G; 6-JOE; Light Cycler® red 610; Light Cycler® red 640; Light Cycler® red 670; Light Cycler® red 705; 7-amino-4-methylcoumarin; 7-aminoactinomycin D (7-AAD); 7-hydroxy-4-methylcoumarin; 9-amino-6-chloro-2-methoxyacridine; 6-methoxy-N-(4-aminoalkyl)quinolinium bromide hydrochloride (AB Q); Acid Fuchsin; ACMA (9-amino-6-chloro-2-methoxyacridine); Acridine Orange; Acridine Red; Acridine Yellow; Acriflavin; Acriflavin Feulgen SITSA; AFPs-AutoFluorescent Protein-(Quantum Biotechnologies); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-lsoThioCyanate); True Blue; TruRed; Ultralite; Uranine B; Uvitex SFC; WW 781; X-Rhodamine; X-Rhodamine-5-(and-6)-Isothiocyanate (5(6)-XRITC); Xylene Orange; Y66F; Y66H; Y66W; YO-PRO-1; YO-PRO-3; YOYO-1; interchelating dyes such as YOYO-3, Sybr Green, Thiazole orange; members of the Alexa Fluor® dye series (from Molecular Probes/Invitrogen) which cover a broad spectrum and match the principal output wavelengths of common excitation sources such as Alexa Fluor 350, Alexa Fluor 405, 430, 488, 500, 514, 532, 546, 555, 568, 594, 610, 633, 635, 647, 660, 680, 700, and 750; members of the Cy Dye fluorophore series (GE Healthcare), also covering a wide spectrum such as Cy3, Cy3B, Cy3.5, Cy5, Cy5.5, Cy7; members of the Oyster® dye fluorophores (Denovo Biolabels) such as Oyster-500, -550, -556, 645, 650, 656; members of the DY-Labels series (Dyomics), for example, with maxima of absorption that range from 418 nm (DY-415) to 844 nm (DY-831) such as DY-415, -495, -505, -547, -548, -549, -550, -554, -555, -556, -560, -590, -610, -615, -630, -631, -632, -633, -634, -635, -636, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL; members of the ATTO series of fluorescent labels (ATTO-TEC GmbH) such as ATTO 390, 425, 465, 488, 495, 520, 532, 550, 565, 590, 594, 610, 611X, 620, 633, 635, 637, 647, 647N, 655, 680, 700, 725, 740; members of the CAL Fluor® series or Quasar® series of dyes (Biosearch Technologies) such as CAL Fluor® Gold 540, CAL Fluor® Orange 560, Quasar® 570, CAL Fluor® Red 590, CAL Fluor® Red 610, CAL Fluor® Red 635, Quasar® 570, and Quasar® 670. In some embodiments, a first photo-detectable label interacts with a second photo-detectable moiety to modify the detectable signal, e.g., via fluorescence resonance energy transfer (“FRET”; also known as Förster resonance energy transfer).

The fluorescent labels utilized by the systems and methods disclosed herein can have different peak absorption wavelengths, for example, ranging from 400 nm to 800 nm. In some embodiments, the peak absorption wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak absorption wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different peak emission wavelength, for example, ranging from 400 nm to 800 nm. In some embodiments, the peak emission wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak emission wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different Stokes shift, for example, ranging from 10 nm to 200 nm. In some embodiments, the stoke shift can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the stoke shift can be at least, or at most, 10, 20, 30, 40, 50, 60, 70, 80, 90, 100, 110, 120, 130, 140, 150, 160, 170, 180, 190, or 200 nm.

Two or more fluorescent labels can have overlapping emission spectra and can be subject to cross-talk. In some embodiments, the distance between the peak emission wavelengths of any two fluorescent labels can vary, for example, ranging from 10 nm to 200 nm. In some embodiments, the distance between the peak emission wavelengths of any two fluorescent labels can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the distance between the peak emission wavelengths of any two fluorescent labels can be at least, or at most, 10, 20, 30, 40, 50, 60, 70, 80, 90, 100, 110, 120, 130, 140, 150, 160, 170, 180, 190, or 200 nm.

Various different types of linkers having different lengths and chemical properties can be used. The term “linker” encompasses any moiety that is useful to connect one or more molecules or compounds to each other, to other components of a reaction mixture, and/or to a reaction site. For example, a linker can attach a reporter molecule or “label” (e.g., a fluorescent dye) to a reaction component. In certain embodiments, the linker is a member selected from substituted or unsubstituted alkyl (e.g., a 2-5 carbon chain), substituted or unsubstituted heteroalkyl, substituted or unsubstituted aryl, substituted or unsubstituted heteroaryl, substituted or unsubstituted cycloalkyl, and substituted or unsubstituted heterocycloalkyl. In one example, the linker moiety is selected from straight- and branched carbon-chains, optionally including at least one heteroatom (e.g., at least one functional group, such as ether, thioether, amide, sulfonamide, carbonate, carbamate, urea and thiourea), and optionally including at least one aromatic, heteroaromatic or non-aromatic ring structure (e.g., cycloalkyl, phenyl). In certain embodiments, molecules that have trifunctional linkage capability are used, including, but are not limited to, cynuric chloride, mealamine, diaminopropanoic acid, aspartic acid, cysteine, glutamic acid, pyroglutamic acid, S-acetylmercaptosuccinic anhydride, carbobenzoxylysine, histine, lysine, serine, homoserine, tyrosine, piperidinyl-1,1-amino carboxylic acid, diaminobenzoic acid, etc. In certain specific embodiments, a hydrophilic PEG (polyethylene glycol) linker is used.

In certain embodiments, linkers are derived from molecules which comprise at least two reactive functional groups (e.g., one on each terminus), and these reactive functional groups can react with complementary reactive functional groups on the various reaction components or used to immobilize one or more reaction components at the reaction site. “Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like.

Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

As used herein, an “optical channel” is a predefined profile of optical frequencies (or equivalently, wavelengths). For example, a first optical channel may have wavelengths of 500 nm-600 nm. To take an image in the first optical channel, one may use a detector which is only responsive to 500 nm-600 nm light, or use a bandpass filter having a transmission window of 500 nm-600 nm to filter the incoming light onto a detector responsive to 300 nm-800 nm light. A second optical channel may have wavelengths of 300 nm-450 nm and 850 nm-900 nm. To take an image in the second optical channel, one may use a detector responsive to 300 nm-450 nm light and another detector responsive to 850 nm-900 nm light and then combine the detected signals of the two detectors. Alternatively, to take an image in the second optical channel, one may use a bandstop filter which rejects 451 nm-849 nm light in front of a detector responsive to 300 nm-900 nm light.

### Additional Notes

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a dicrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A system for identifying a nucleotide in a nucleic acid sequence bound to a substrate, comprising:
a first detector configured to detect a first range of wavelengths of light;
a second detector configured to detect a second range of wavelengths of light;
a light source comprising a laser or a light-emitting diode which outputs light at an optical frequency; and
a processor configured to:
generate light at the optical frequency to stimulate an emission from the nucleic acid sequence on the substrate; and
identify a nucleotide in the nucleic acid sequence based on whether the emission is received by the first detector, the second detector, both the first and second detectors, or neither the first nor second detector.

2. The system of claim 1, further comprising:
a first nucleotide coupled to a first fluorescent label;
a second nucleotide coupled to a second fluorescent label;
a third nucleotide coupled to a third fluorescent label; and
a fourth nucleotide coupled to no fluorescent label,
wherein the light source is configured to:
excite the first fluorescent label to emit light to be detectable by the first detector;
excite the second fluorescent label to emit light to be detectable by the second detector; and
excite the third fluorescent label to emit light to be detectable by both the first and second detectors.

3. The system of claim 2, wherein the fluorescent labels are selected from the group consisting of polymethine derivatives, coumarin derivatives, benzopyran derivatives, and chromenoquinoline derivatives.

4. The system of claim 2, wherein the fluorescent labels are selected from the group consisting of

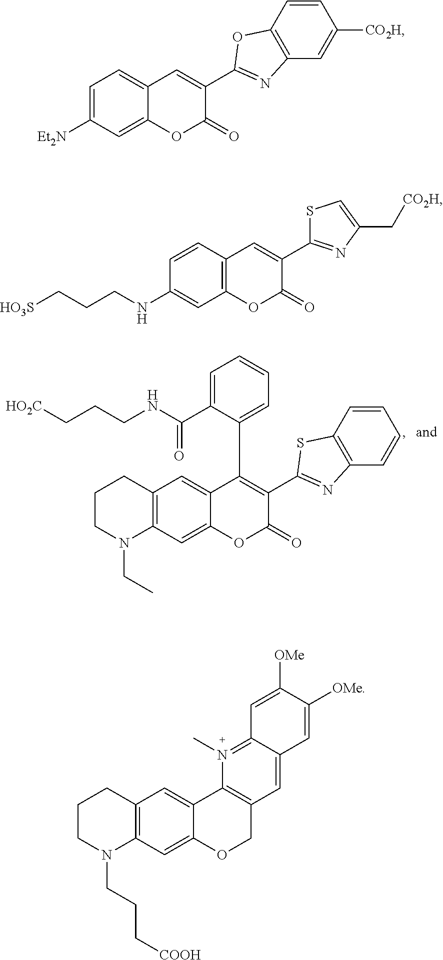

5. The system of claim 2, further comprising an additional first nucleotide coupled to no fluorescent label.

6. The system of claim 2, further comprising an additional first nucleotide coupled to an alternative fluorescent label, wherein the alternative fluorescent label cannot be excited by the light source to emit light to be detectable by the first detector.

7. The system of claim 2, further comprising an additional first nucleotide coupled to an alternate fluorescent label, wherein the alternate fluorescent label can be excited by the light source to emit light to be detectable by the first detector, and wherein the alternate fluorescent label emits dimmer light as compared to the first fluorescent label.

8. The system of claim 6, wherein the alternative fluorescent label is

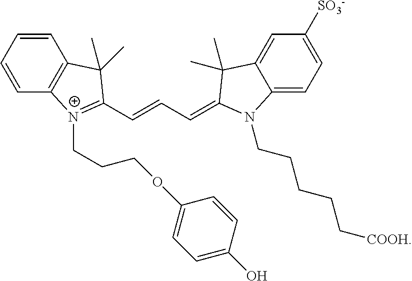

9. The system of claim 6, wherein the alternative fluorescent label and the first fluorescent label have different fluorescence emission spectra.

10. The system of claim 2, wherein the first fluorescent label has a Stokes shift between 20 nm-50 nm, the second fluorescent label has a Stokes shift between 100 nm-130 nm, and the third fluorescent label has a Stokes shift between 60 nm-90 nm.

11. The system of claim 2, wherein the first fluorescent label is not detectable by the second detector, and wherein the second fluorescent label is not detectable by the first detector.

12. The system of claim 2, wherein the first fluorescent label is also detectable by the second detector, or wherein the second fluorescent label is also detectable by the first detector.

13. The system of claim 1, wherein the first range of wavelengths and the second range of wavelengths do not overlap.

14. The system of claim 1, wherein the optical frequency corresponds to a wavelength in a predefined range of wavelengths of light, wherein the predefined range comprises at least one wavelength that is shorter than all of the wavelengths in the first range and in the second range.

15. The system of claim 14, wherein the predefined range comprises 405 nm-460 nm.

16. The system of claim 1, wherein the optical frequency corresponds to a wavelength in a predefined range of wavelengths of light, wherein the predefined range comprises at least one wavelength that is longer than some of the wavelengths in the first range or in the second range.

17. The system of claim 2, wherein the light source is configured to excite the fluorescent labels by two-photon absorption processes.

18. The system of claim 1, wherein the detectors comprise complementary metal-oxide-semiconductor image sensors, charge-coupled device image sensors, photomultiplier tubes, photodiodes, or any combination thereof.

19. The system of claim 1, further comprising one or more optical filter materials, one or more diffraction gratings, one or more light dispersing elements, or any combination thereof.

20. The system of claim 2, further comprising a polymerase configured to replicate or transcribe a portion of the nucleic acid sequence by incorporating the nucleotides.

21. The system of claim 1, wherein the substrate comprises a plurality of chemically functionalized regions, a plurality of cavities, a plurality of optical resonators, a plurality of optical waveguides, or any combination thereof.

22. The system of claim 2, wherein the nucleotides and the fluorescent labels are coupled by cleavable linkers.

23. The system of claim 2, wherein the nucleotides are selected from the group consisting of an analog of dGTP, an analog of dTTP, an analog of dUTP, an analog of dCTP, and an analog of dATP.

24. The system of claim 2, wherein the first nucleotide is a first reversibly blocked nucleotide triphosphate (rbNTP), the second nucleotide is a second rbNTP, the third nucleotide is a third rbNTP, and the fourth nucleotide is a fourth rbNTP.

25. The system of claim 24, wherein the four rbNTPs are selected from the group consisting of rbATP, rbTTP, rbUTP, rbCTP, and rbGTP.

26. The system of claim 24, wherein each of the four rbNTPs comprises a modified base and a reversible terminator 3′ blocking group.

27. A method for determining the sequence of a polynucleotide, comprising:
emitting light at an optical frequency from a light source onto a polynucleotide;
determining if the polynucleotide has a bound fluorescent label which fluoresces at a first wavelength of light, a second wavelength of light, both the first and second wavelengths of light, or has no fluorescence; and
identifying the sequence of the polynucleotide based on whether there is a detectable emission at the first wavelength of light, the second wavelength of light, both the first and second wavelengths of light, or has no fluorescence.

28. The method of claim 27, wherein determining if the polynucleotide has a bound fluorescent label comprises:
determining if a first nucleotide is coupled to a first fluorescent label;
determining if a second nucleotide is coupled to a second fluorescent label;
determining if a third nucleotide is coupled to a third fluorescent label; and
determining if a fourth nucleotide is coupled to no fluorescent label.

29. The method of claim 27, wherein the bound fluorescent label is selected from the group consisting of polymethine derivatives, coumarin derivatives, benzopyran derivatives, and chromenoquinoline derivatives.

30. The method of claim 27, wherein the first fluorescent label has a Stokes shift between 20 nm-50 nm, the second fluorescent label has a Stokes shift between 100 nm-130 nm, and the third fluorescent label has a Stokes shift between 60 nm-90 nm.
